SEQUENCE LISTING (2 sequences)

SEQ ID NO: 1 - protein, 360 aa, Homo sapiens
MLSTSRSRFIRNTNESGEEVTTFFDYDYGAPCHKFDVKQIGAQLLPPLYSLVFIFGFVGNMLVVLILINCKKLKCLTDIYLLNLAISDLLFLITLPLWAHSAANEWVFGNAMCKLFTGLYHIGYFGGIFFIILLTIDRYLAIVHAVFALKARTVTFGVVTSVITWLVAVFASVPGIIFTKCQKEDSVYVCGPYFPRGWNNFHTIMRNILGLVLPLLIMVICYSGILKTLLRCRNEKKRHRAVRVIFTIMIVYFLFWTPYNIVILLNTFQEFFGLSNCESTSQLDQATQVTETLGMTHCCINPIIYAFVGEKFRRYLSVFFRKHITKRFCKQCPVFYRETVDGVTSTNTPSTGEQEVSAGL

SEQ ID NO: 2 - protein, 7 aa, Artificial Sequence
  Synthetic peptide
MLVVLIL

(19) United States
(12) Patent Application Publication
(10) Pub. No.: US 2014/0336127 A1
(43) Pub. Date: Nov. 13, 2014

(54) Stabilized Peptide Helices For Inhibiting Dimerization Of Chemokine C Motif Receptor 2 (CCR2)

(71) Applicant: Yissum Research Development Company of the Hebrew University of Jerusalem Ltd., Jerusalem (IL)

(72) Inventors: Chaim Gilon, Jerusalem (IL); Maya Ratner-Hurevich, Jerusalem (IL); Mattan Hurevich, Jerusalem (IL)

(21) Appl. No.: 14/339,027

(22) Filed: Jul. 23, 2014

Related U.S. Application Data
(63) Continuation of application No. PCT/IL2013/050060, filed on Jan. 21, 2013.
(60) Provisional application No. 61/589,405, filed on Jan. 23, 2012.

(51) Int. Cl. A61K 47/48; C07K 9/00; C07K 7/06

(52) U.S. Cl. CPC A61K 47/48023; C07K 7/06; C07K 9/001; A61K 47/48092; A61K 47/48215
USPC 514/17.9; 530/329; 530/322

(57) ABSTRACT

Peptide helices stabilized by backbone cyclization which are capable of inhibiting dimerization of the Chemokine (C-C motif) receptor 2 (CCR2), as well as pharmaceutical compositions including such backbone cyclized peptide helices. Use of pharmaceutical compositions and peptide helices in treatment of Multiple Sclerosis (MS) and other diseases associated with CCR2 activation.

### FIELD OF THE INVENTION

The present invention relates to inhibition of dimerization of the chemokine C motif receptor 2 (CCR2) by peptide helices stabilized by backbone cyclization, pharmaceutical compositions comprising these compounds, and methods for using them in treatment of multiple sclerosis and other diseases and disorders associated with activation of the CCR2 receptor.

### BACKGROUND OF THE INVENTION

Multiple sclerosis (MS) is an autoimmune inflammatory demyelinating disease of the central nervous system (CNS). MS affects mainly young adults and it is the leading cause of neurological disability in this age group. The course of the MS is either relapsing and remitting or progressive. During the relapses of the disease, autoimmune, anti-myelin reactive lymphocytes are produced, activated and recruited from the peripheral immune system, enter the CNS and attack the myelin components, inducing neurological deficits which depend on the area of the white matter of the CNS that is affected each time (i.e. loss of vision, motor paralysis, instability of gait, problems in coordination of movements, loss of sphincters control, sensory disturbances etc). Despite dramatic improvement during the last decades, in the diagnostic tools for MS (basically due to the widespread availability of brain and spinal MRI), understanding of the basic etiology of the disease remains limited. Fully effective control of the disease activity and progression and the repair of damaged myelin are key objectives for current and future investigators. Based on the widely accepted autoimmune pathogenetic model, the current treatment options for MS include various modalities that downregulate or modulate the inflammatory process and the immune anti-myelin responses. Acute attacks (relapses) of MS are typically treated with glucocorticoids. Patients with relapsing-remitting MS who have current disease activity manifested by clinical symptoms or active new MRI lesions are treated with other, long-term acting, immunomodulatory drugs, such as interferon beta (Avonex®, Rebif®, Betaseron®), glatiramer acetate (Copaxone®), fingolimod and the chemotherapeutic agent mitoxanthrone (Compston, A.; Coles, A., Multiple sclerosis. Lancet 2008, 372, (9648), 1502-17). Almost all of these drugs are administered with injections and are associated with various adverse effects which both limit their ease of use for long periods of time. In addition, all of these treatments are partially effective and can only reduce the relapse and progression rates of MS by approximately 30%.

Chemokine (C-C motif) receptor 2 (CCR2) is a receptor for monocyte chemoattractant protein-1 (CCL2), a chemokine which specifically mediates monocyte chemotaxis. CCR2 is a key player in the recruitment of autoimmune myelin-reactive lymphocytes into the CNS, thus, its inhibition may prevent the migration of these inflammatory cells to the brain or spinal cord, providing thus a novel therapeutic approach for MS. Dimerization of CCR2 was associated with immune cell recruitment that takes place in immune diseases such as MS (Allen et al., Annu Rev Immunol 2007, 25, 787-820). The essential role of CCR2 dimerization in experimental autoimmune encephalomyelitis (EAE) the animal model for MS, makes it an attractive target for developing drug leads for treatment of this disease (Mahad et al., Brain 2006, 129, (Pt 1), 212-23). Studies performed on CCR2-knockout mice showed that (EAE) can not be induced in CCR2-deficient mice, probably due to the inability of immune cells to migrate to the CNS (Fife et al., J Exp Med 2000, 192, (6), 899-905; Siebert et al., J Neuroimmunol 2000, 110, (1-2), 177-85).

CCR2 dimerization is mediated through the interaction of the receptor with the corresponding chemokine, monocyte chemo-attractant protein-1 (CCL2 or MCP-1). Studies involving MCP-1 induced CCR2 dimerization indicated that few regions in the transmembrane part of the protein interact with parallel regions in other receptors to form either homodimers or heterodimers. Although peptides derived from the dimerization site proved to have an inhibitory effect on CCR2 dimerization, they lacked crucial pharmacological properties necessary to become drug leads. Since the three dimensional structure of CCR2 has not been resolved, rational design of macrocyclic CCR2 dimerization blocker drug leads using standard means (NMR, X-Ray, computation) is extremely challenging.

Helix mimetics by linear peptides is not feasible since they do not form the desired helical structure in solution (Haridas, V., Eur J O Chem 2009, (30), 5112-5128). Peptide stabilization through cyclization is being used to induce helical structure to peptides. In these methods, a covalent bond is added artificially to the peptide sequence to stimulate helix formation and substitute the native hydrogen bond. In previous works, the hydrogen bond was replaced by “natural” covalent bonds such as disulfide or amide bonds or by “non-natural” bonds such as hydrazone or olefins (Patgiri et al., Nat Protoc 2010, 5, (11), 1857-65; Garner, J.; Harding, M. M., Organic & Biomolecular Chemistry 2007, 5, (22), 3577-3585) to form cyclic helix mimetics. Helix mimetic cyclic peptides displayed remarkable pharmacological properties like stability in water and improved bioactivity but suffer from disadvantages such as need to change the peptide sequence by replacing or adding amino acids.

Backbone cyclization (BC) was already proved to be a valuable tool in methodological conversion of active sites of proteins to cyclic peptides and even to small macrocycles (Hurevich et al., Bioorg Med Chem 2010, 18, (15), 5754-5761; Hayouka et al., Bioorg Med Chem 2010, 18, (23), 8388-8395; Hess et al., J Med Chem 2008, 51, (4), 1026-34). The BC method is used to introduce global constraints to active peptides. It differs from other cyclization methods since it utilizes non-natural building blocks for cycle anchors, mainly N-alkylated amino acids. BC proved superior to other stabilization methods since the resultant peptides had defined structures that led to better selectivity (Gazal et al., J Med Chem 2002, 45, (8), 1665-71; WO 99/65508) and improved pharmacological properties. The use of BC enables a combinatorial approach called “cycloscan”. It was used for generating and screening BC peptide libraries to find lead peptides that overlap with the bioactive conformation. In a cycloscan, all the peptides in the library bear the same sequence but differ from each other in other parameters that constraint the conformational space. Screening the library allows an iterative evaluation of the effect of chemical modifications on the structural properties and biological function. Changing the ring size and ring chemistry proved to be the most convenient modification to perform in cycloscan and has been used to synthesize small- and medium-sized peptide libraries. However, identifying the correct anchor position in the cyclic peptide is a challenging step that can be done only when sufficient preliminary information is available, which is not the case for CCR2.

There is an unmet need for metabolically stable, tissue permeable, preferably orally bioavailable and more effective therapeutic modalities for MS.

### SUMMARY OF THE INVENTION

The present invention provides compounds designed to serve as Chemokine (C-C motif) receptor 2 (CCR2) peptide-based stabilized helices that mimic the helical dimerization region of the receptor. These novel compounds are capable of inhibiting dimerization of the receptor (from which they are derived. The present invention further provides compounds, pharmaceutical compositions and methods of treating MS and other conditions associated with activation of CCR2. Also provided are methods for stabilizing peptide helices and identifying cyclic peptides which inhibit the CCR2 receptor activation.

Although insertion of N-alkylated amino acid residue, such as N-methyl-Alanine or Proline residues into the sequence of a peptide helix is known to breaks the helical structure, it was unexpectedly found, that insertion of N^{α}-ω-functionalized derivative of an amino acid residue within a helical structure of CCR2 derived peptide, to achieve backbone cyclization, do not interrupt the helix but rather stabilize it. It is therefore disclosed herein for the first time that backbone cyclization can be used to stabilize helical structures of a peptide to form biological active helical peptide mimetics.

The present invention provides, according to one aspect, a peptide helix mimetic of 5-30 amino acids, or an analog thereof, comprising a sequence derived from the G-protein coupled receptor Chemokine (C-C motif) receptor 2 (CCR2), wherein the helix is stabilized by backbone cyclization.

According to some embodiments, the stabilized peptide helix mimetic consists of 5-15 amino acids. According to other embodiments the stabilized peptide helix mimetic consists of 5-12 amino acid residues. According to yet other embodiments, the stabilized peptide helix mimetic consists of 7-10, 7-9 or 7-8 amino acid residues. Each possibility represents a separate embodiment of the present invention.

According to a specific embodiment, the chemokine CCR2 receptor is human CCR2b subtype (SEQ ID NO: 1) of the sequence

| | |
|---|---|
| 1 | mlstsrsrfi rntnesgeev ttffdydyga pchkfdvkqi |
| | gaqllpplys lvfifgfvgn |
| 61 | mlvvlilinc kklkcltdiy llnlaisdll flitlplwah |
| | saanewvfgn amcklftgly |
| 121 | higyfggiff iilltidryl aivhavfalk artvtfgvvt |
| | svitwlvavf asvpgiiftk |
| 181 | cqkedsvyvc gpyfprgwnn fhtimrnilg lvlpllimvi |
| | cysgilktll rcrnekkrhr |
| 241 | avrviftimi vyflfwtpyn ivillntfqe ffglsncest |
| | sqldqatqvt etlgmthcci |
| 301 | npiiyafvge kfrrylsvff rkhitkrfck qcpvfyretv |
| | dgvtstntps tgeqevsagl |

According to some embodiments, the peptide sequence is derived from a transmembrane segment of CCR2. According to some specific embodiments, the peptide sequence is derived from the sequence of the transmembrane 1 (TM-1) domain of CCR2.

According to some embodiments, the sequence derived from TM-1 comprises at least five amino acids of the sequence MLVVLIL (SEQ ID NO: 2), corresponding to amino acid residues 61-67 of human CCR2b, or an analog thereof comprising at least one amino acid substitutions, deletions or additions to SEQ ID NO: 2.

According to some embodiments, the analog comprises modification selected from the group consisting of: 1-2 deletions of amino acids, 2-3 substitutions of amino acids, 1-8 additions of amino acids, addition of a linker, and combinations thereof. Each possibility represents a separate embodiment of the present invention.

Peptide helices according to the present invention are stabilized by connecting two amino acid residues of the helix, using a backbone cyclization, namely, covalently connecting at least one amino acid residue in the helix sequence, which was substituted with a N^{α}-ω-functionalized or an C^{α}-ω-functionalized derivative of amino acid residue, with a moiety selected from the group consisting of: another N^{α}-ω-functionalized or an C^{α}-ω-functionalized derivative of amino acid residue, with the side chain of an amino acid in the peptide sequence, or with one of the peptide terminals.

Any covalent bond may be used to connect the anchoring positions of the CCR2 helix sequence using backbone cyclization. According to some embodiments, the backbone cyclization covalent bond is selected from the group consisting of: amide bond, disulfide bond, and urea bond. According to some particular embodiments, the backbone cyclization bond used for stabilizing the helix of the invention is a urea bond.

According to some embodiments, the cycle anchor positions are identified using a combinatorial “cycloscan” approach.

According to some embodiments, CCR2 helices are stabilized by covalently connecting positions i,i+4, i,i+7 or i,i+3 of the peptide sequence using backbone cyclization.

The present invention provides, according to some specific embodiments, a synthetic peptide helix of 5-15 amino acids, comprising the sequence MLVVLIL (SEQ ID NO: 2) or an analog of SEQ ID NO: 2 comprising at least one amino acid deletion, addition or substitution, and wherein the helix structure is stabilized by covalently connecting at least one N^{α}-ω-functionalized derivative of amino acid residue added to the sequence, or substituted an amino acid residue in the sequence, with a moiety selected from the group consisting of: another N^{α}-ω-functionalized derivative of amino acid residue; the side chain of an amino acid in the peptide sequence; and one of the peptide terminals, to form a backbone cyclized peptide.

According to some embodiments, the peptide helix further comprises a permeability enhancing moiety connected to the peptide directly or through a linker or spacer.

The permeability-enhancing moiety may be connected to any location of the peptide sequence. According to some embodiments, the permeability-enhancing moiety is a hydrophilic moiety. According to some specific embodiments, the hydrophilic moiety is connected to the N-terminus of the peptide helix. According to other embodiments, the hydrophilic moiety is part of the backbone cyclization bridge. Each possibility represents a separate embodiment of the present invention.

According to some embodiments, the peptide helix consists of 7-12 amino acid residues. According to other embodiments, the peptide helix consists of 7-10, 7-9 or 7-8 amino acid residues. According to yet other embodiments, the peptide helix consists of 5-10, 5-9, 5-8 or 5-7 amino acid residues. Each possibility represents a separate embodiment of the present invention.

According to some embodiments, the helix structure is stabilized by covalently connecting one N^{α}-ω-functionalized derivative of amino acid residue added to the sequence, or substituted an amino acid residue in the sequence, with another N^{α}-ω-functionalized derivative of amino acid residue in the sequence.

According to some embodiments, the analog of SEQ ID NO: 2 comprises modification selected from the group consisting of: 1-2 deletions of amino acids, 2-3 substitutions of amino acids, 1-8 additions of amino acids, addition of a linker, and combinations thereof. Each possibility represents a separate embodiment of the present invention.

According to some embodiments, the building units are connected by a bond selected from the group consisting of: urea bond, thiourea bond, amide bond, disulfide bond and guanidino group.

According to some particular embodiments, the bridge is selected from the group consisting of: urea bridge, thiourea bridge and guanidino bridge.

According to some embodiments, at least one N^{α}-ω-functionalized amino acid residue used for cyclization is located at position 4 of the peptide sequence (numbered from the N terminus of the peptide). According to particular embodiments, backbone cyclization is between positions (numbered from the peptide N-terminus) selected from the group consisting of: 4-7 and 1-4. According to other embodiments, backbone cyclization is between position 4 and the C-terminus or N-terminus of the peptide. Each possibility represents a separate embodiment of the present invention.

According to some specific embodiments, the synthetic peptide helix of 5-15 amino acids stabilized by backbone cyclization is according to Formula I:

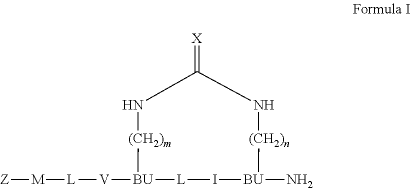

wherein m is an integer of 2-6; n is an integer of 2-6; X is selected from the group consisting of: O, S and NH; Z is selected from the group consisting of: hydrogen, a carbohydrate moiety, a hydrophilic moiety, a polyethylene glycol (PEG), and a triglycerol; and BU designates a N^{α}-ω-functionalized amino acid residue.

According to some specific embodiments, m is 2 and n is 4. According to other embodiments, m is 4 and n is 2.

According to some specific embodiments BU designates a N^{α}-ω-functionalized Glycine residue.

According to other embodiments BU designates a N^{α}-ω-functionalized residue of a natural or synthetic amino acid other than Glycine.

According to some embodiments Z is a hydrophilic moiety. According to some particular embodiments the hydrophilic moiety is selected from the group consisting of: 1-5 hydrophilic amino acid residues, a gauinidino group, a carbohydrate moiety.

According to some embodiments, the hydrophilic moiety comprises 1-3 Arginine residues or a guanidino-containing moiety.

According to some particular embodiments, the carbohydrate moiety is a glucose or trehalose residue or a derivative thereof.

According to other embodiments, Z is selected from a polyethylene glycol (PEG) moiety and a triglycerol moiety.

The moiety Z may be connected directly to the peptide sequence or, according to other embodiments, through a linker or spacer.

Each possibility represents a separate embodiment of the present invention.

According to a particular embodiment, the peptide helix is stabilized by urea backbone cyclization to form a structure selected from the group consisting of:

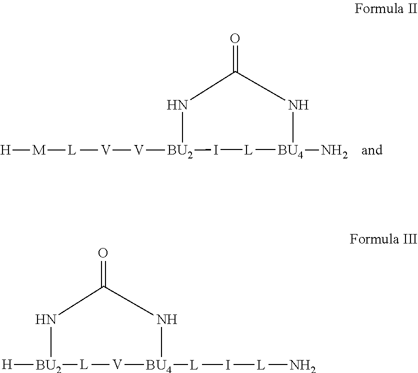

wherein, BU designates a N^{α}-ω-functionalized amino acid residue of the formula:

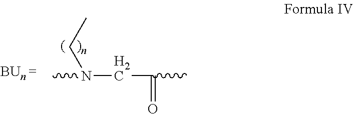

According to some embodiments the backbone stabilized peptide mimetic is M3D-1 having the schematic structure M-L-V-BU-L-I-BU-NH₂ wherein BU designates N^{α}-ω-functionalized Glycine residue and wherein the two BUs are connected through urea bond to form the compound of Formula V:

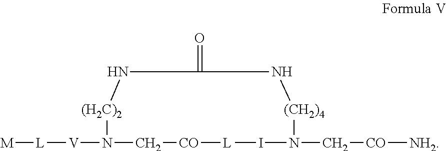

Combinations of substitutions, additions and bridge modifications described with respect to specific embodiments, as well as combination of such substitutions, additions or modifications with deletion of 1-2 amino acid residues, are also within the scope of the present invention.

According to some particular embodiments, the backbone cyclized peptide helix mimetic, is selected form the group consisting of:

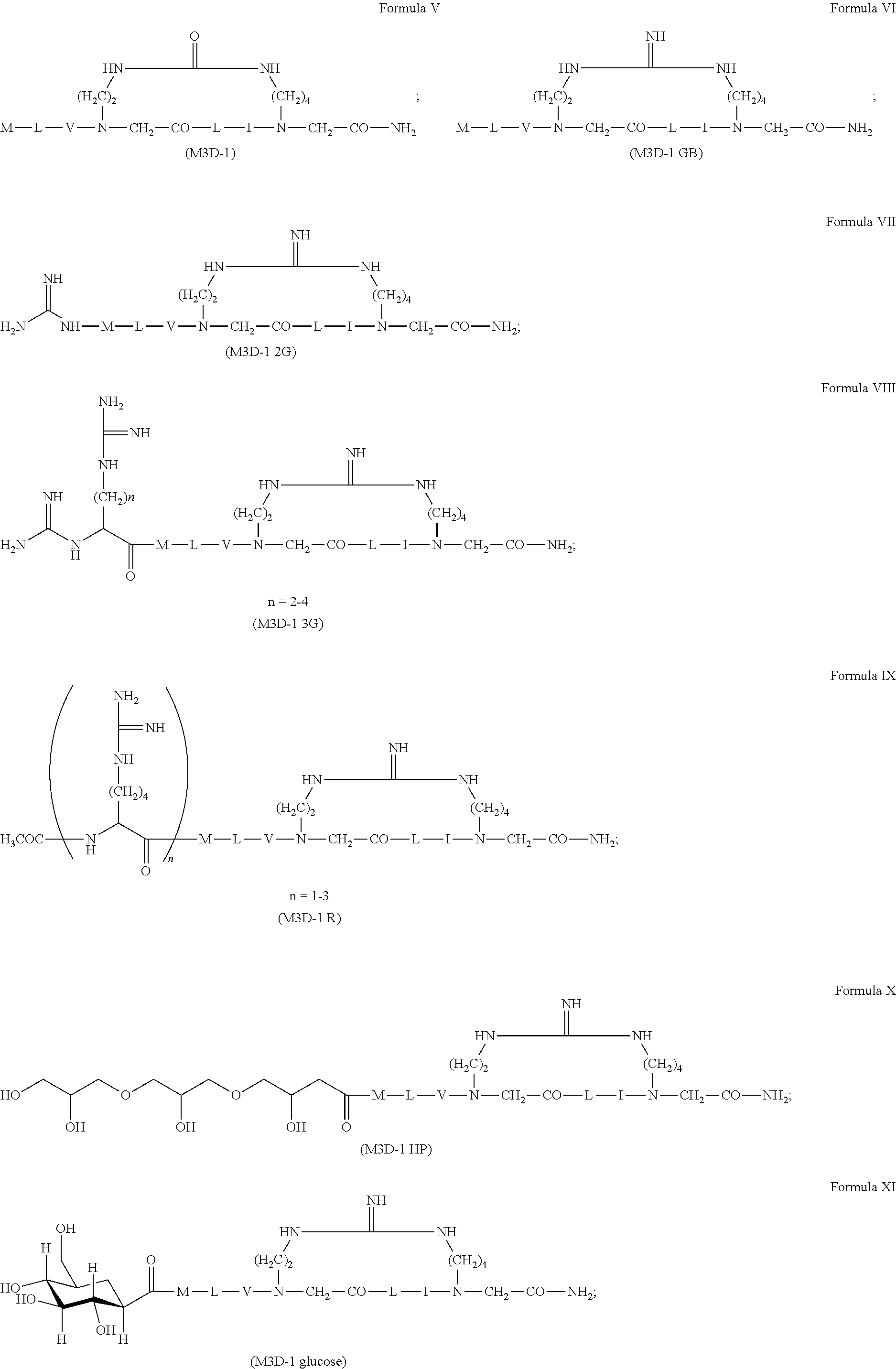

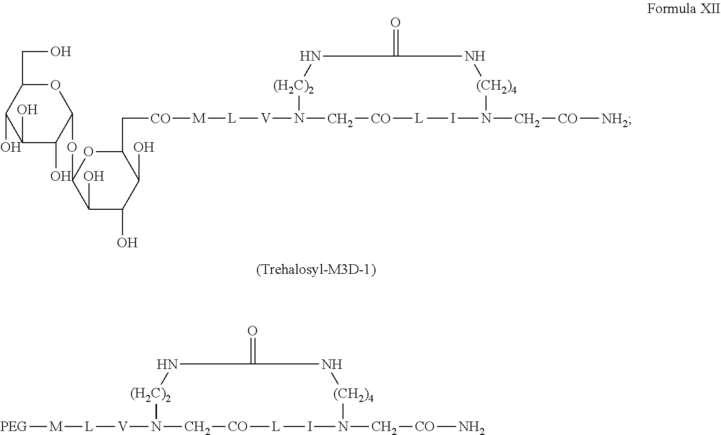

Formula XIII (PEGylated M3D-1) wherein PEG is polyethylene glycol; and

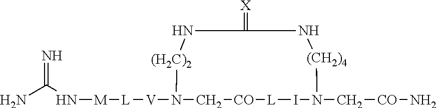

Formula XIV (M3D-1 with bridge chemistries and guanidino alpha amine modification, wherein X is O, N or S).

Pharmaceutical compositions comprising at least one CCR2 peptide helix stabilized by backbone cyclization are provided according to another aspect of the present invention, as well as their use in treatment of diseases and disorders associated with CCR2 expression. According to a specific embodiment, the disease of disorder associated with CCR2 expression is MS.

Pharmaceutical compositions for use in accordance with the present invention may be formulated in conventional manner using one or more physiologically acceptable carriers comprising excipients and auxiliaries, which facilitate processing of the active compounds into preparations which, can be used pharmaceutically. Proper formulation is dependent upon the route of administration chosen.

According to some embodiments, the pharmaceutical compositions are formulated for oral administration.

According to other embodiments, the pharmaceutical compositions are formulated for parenteral administration.

According to some embodiments the scaffold of the stabilized helix confers permeability of the molecule. According to other embodiments the molecule comprises a permeability enhancing moiety, connected to the peptide.

Any moiety known in the art to actively or passively facilitate or enhance permeability of the compounds into cells may be used for conjugation with the molecules of the present invention. Non-limitative examples include: moieties which may have cell-membrane receptors or carriers, such as steroids, vitamins and sugars, natural and non-natural amino acids and transporter peptides. Hydrophobic helices according to the present invention may be preferably conjugated with hydrophilic moieties to enhance permeability.

According to some embodiments the formulation further comprises an excipient, carrier or diluent suitable for oral administration. Suitable pharmaceutically acceptable excipients for use in this invention include those known to a person ordinarily skilled in the art such as diluents, fillers, binders, disintegrants and lubricants. Diluents may include but not limited to lactose, microcrystalline cellulose, dibasic calcium phosphate, mannitol, cellulose and the like. Binders may include but not limited to starches, alginates, gums, celluloses, vinyl polymers, sugars and the like. Lubricants may include but not limited to stearates such as magnesium stearate, talc, colloidal silicon dioxide and the like.

The present invention provides, according to another aspect, a method of prevention, alleviation or treatment of a disease or disorder associated with expression of CCR2 comprising administering to a subject in need thereof, a pharmaceutically active amount of stabilized helical peptide according to the invention. According to certain embodiments the disease or disorder associated with CCR2 expression is MS. According to some embodiments the administration route is selected from the group consisting of: orally, topically, intranasally, subcutaneously, intramuscularly, intravenously, intra-arterially, intraarticulary, intralesionally or parenterally.

Use of a stabilized peptide helix according to the invention for preparation of a medicament for prevention or treatment of disease or condition associated with CCR2 is also within the scope of the present invention.

According to certain embodiments the disease or condition associated with CCR2 is MS. According to some embodiments, the MS is selected from the group consisting of relapsing remitting MS, secondary progressive MS, primary progressive MS, and progressive relapsing MS.

The present invention provides, according to yet another aspect, a method of stabilizing peptide helices in a favored conformation to be used as inhibitors or activators of a signal transduction of CCR2, the method comprises synthesizing backbone cyclization peptides derived from CCR2 having different anchoring positions and bridge lengths, testing the backbone cyclized peptides for an activity and optimizing the bridge location and size if necessary.

According to some embodiments, backbone cyclization is performed by a bond selected from the group consisting of: amide, disulfide and urea. According to a particular embodiment, backbone cyclization is performed by urea bonds.

According to some embodiments, backbone cyclization is performed between at least one N-alpha alkylated amino acid residue of the helix sequence and another moiety selected from the group consisting of: additional N-alpha alkylated amino acid residue of the helix sequence, an amino acid side chain of the helix sequence, and one of the peptide terminals.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE FIGURES

FIG. 1 is a schematic representation of CCR5 and CCR2 dimerization regions.

FIG. 2 describes inhibition of MCP-1 mediated monocytes migration of hCCR2b peptides derived from the transmembrane-1 domain (TM-1). No MCP-1 (dotted); no peptide (black); hCCR2 truncated peptides 63-67, 64-67 and 62-67 (gray); andhCCR2(61-67) peptide (striped).

FIGS. 3A-3C show rational design of CCR2 dimerization blocker. (FIG. 3A) Modes of helix stabilization; (FIG. 3B) i to i+3 urea backbone cyclic model; (FIG. 3C) schematic presentation of TM-1 mimetic ring position scan (helix walk).

FIG. 4 depicts urea backbone cyclic ring positions scan of CCR2 dimerization site hCCR2(61-67) segment MLVVLIL. BU designates —N—CH₂(CH₂)ₙ—CO—.

FIG. 5 describes the synthesis of M3D-1 using the following conditions: a) 20% piperidine, microwave b) Fmoc-AA-OH, HATU, DIPEA, microwave c) Pd(PPh3)4 (0), PhSiH3 d) BTC, DIPEA e) TFA, TIPS, TDW, EDT.

FIG. 6A represents Circular dichroism (CD) screening of M3D library. From top: M3D-5, M3D-3, M3D-4, M3D-1; FIG. 6B represents the influence of TFE percentage on the CD spectra of M3D-1:1% TFE, 5% TFE and 10% TFE.

FIG. 7 describes inhibition of MCP-1 but not SDF-1 mediated monocyte migration by the backbone cyclized helix peptide mimetic M3D-1. Migration without chemokine (Dots), MCP-1-mediated migration (stripes), SDF-1-mediated migration (Crosshatch).

FIG. 8 depicts the structures of the backbone cyclized helix peptide mimetic M3D-1 (compound A), the bridge chemistry BM3D-1 (compounds B and C) and bridge position BP-M3D-1 (compounds D, E, F) based on M3D-1.

FIG. 9 shows the results of metabolic stability BBMV assay testing degradation by intestinal peptidases of the compound M3D-1, in comparison to the linear CCR2b(61-67) peptide.

### DETAILED DESCRIPTION OF THE INVENTION

In the search for a CCR2 dimerization inhibitor, identifying the appropriate cyclization points for stabilizing a helical structure demanded a new synthetic approach. Ring position library synthesis is tedious since it cannot be done in a combinatorial manner. An optimal strategy for ring position scan should include the use of appropriate building blocks and a matching cyclization method that allow varying the ring location. The combination of backbone cyclization (BC) and helix mimetics is appealing since it gives a new dimension to helix mimetics and can directly lead to cyclic peptides with “drug-like” properties. Applying backbone cyclization for stabilizing a helical structure of a peptide is not obvious since incorporation of an N-alkylated amino acid residue, such as Proline within a peptide sequence is know to results in breakage of the helical structure.

A novel ring position screening (helix walk) by urea backbone cyclic peptides was utilized herein, which aim to mimic CCR2 helix motif. The helix walk approach was used to discover the correct position for anchoring the cyclization moieties in order to mimic the CCR2 dimerization site. The presented strategy enabled systematic screening for the appropriate ring anchor position. The helical structure of some of the compounds has been confirmed. The compound M3D-1, for example, blocks specific CCR2 chemokine mediated cell migration (in the low micro-molar range) and is cell permeable and oral available and therefore represents improvement over most of current treatments of MS which are administered by repeated injections. Using this method, active urea backbone cyclic helix peptide mimetics were synthesized, which form stable helical structure and proved to block MCP-1-induced monocyte migration.

### Cyclic Peptides and Backbone Cyclization

Cyclization of peptides has been shown to be a useful approach in developing diagnostically and therapeutically useful peptidic and peptidomimetic agents. Cyclization of peptides reduces the conformational freedom of these flexible, linear molecules, and often results in higher receptor binding affinities by reducing unfavorable entropic effects. Because of the more constrained structural framework, these agents are more selective in their affinity to specific receptor cavities. By the same reasoning, structurally constrained cyclic peptides confer greater stability against the action of proteolytic enzymes (Humphrey, et al., 1997, Chem. Rev., 2243-2266).

Methods for cyclization can be classified into cyclization by the formation of the amide bond between the N-terminal and the C-terminal amino acid residues, and cyclizations involving the side chains of individual amino acids. The latter method includes the formation of disulfide bridges between two-thio amino acid residues (cysteine, homocysteine), the formation of lactam bridges between glutamic/aspartic acid and lysine residues, the formation of lactone or thiolactone bridges between amino acid residues containing carboxyl, hydroxyl or mercapto functional groups, the formation of thioether or ether bridges between the amino acids containing hydroxyl or mercapto functional groups and other special methods. Lambert, et al., reviewed variety of peptide cyclization methodologies (J. Chem. Soc. Perkin Trans., 2001, 1:471-484).

Backbone cyclization is a general method by which conformational constraint is imposed on peptides. In backbone cyclization, atoms in the peptide backbone (N and/or C) are interconnected covalently to form a ring. Backbone cyclized analogs are peptide analogs cyclized via bridging groups attached to the alpha nitrogens or alpha carbonyl of amino acids. In general, the procedures utilized to construct such peptide analogs from their building units rely on the known principles of peptide synthesis; most conveniently, the procedures can be performed according to the known principles of solid phase peptide synthesis. During solid phase synthesis of a backbone cyclized peptide the protected building unit is coupled to the N-terminus of the peptide chain or to the peptide resin in a similar procedure to the coupling of other amino acids. After completion of the peptide assembly, the protective group is removed from the building unit's functional group and the cyclization is accomplished by coupling the building unit's functional group and a second functional group selected from a second building unit, a side chain of an amino acid residue of the peptide sequence, and an N-terminal amino acid residue.

As used herein the term “backbone cyclic peptide” or “backbone cyclic analog” refers to a sequence of amino acid residues wherein at least one nitrogen or carbon of the peptide backbone is joined to a moiety selected from another such nitrogen or carbon, to a side chain or to one of the termini of the peptide. According to specific embodiment of the present invention the peptide sequence is of 5 to 15 amino acids that incorporates at least one building unit, said building unit containing one nitrogen atom of the peptide backbone connected to a bridging group comprising an amide, thioether, thioester, disulfide, urea, carbamate, or sulfonamide, wherein at least one building unit is connected via said bridging group to form a cyclic structure with a moiety selected from the group consisting of a second building unit, the side chain of an amino acid residue of the sequence or a terminal amino acid residue. Furthermore, one or more of the peptide bonds of the sequence may be reduced or substituted by a non-peptidic linkage.

A “building unit” (BU) indicates a N^{α}-ω-functionalized or an C^{α}-ω-functionalized derivative of amino acids. Use of such building units permits different length and type of linkers and different types of moieties to be attached to the scaffold. This enables flexible design and easiness of production using conventional and modified solid-phase peptide synthesis methods known in the art.

In general, the procedures utilized to construct backbone cyclic molecules and their building units rely on the known principles of peptide synthesis and peptidomimetic synthesis; most conveniently, the procedures can be performed according to the known principles of solid phase peptide synthesis. Some of the methods used for producing N building units and for their incorporation into peptidic chain are disclosed in U.S. Pat. Nos. 5,811,392; 5,874,529; 5,883,293; 6,051,554; 6,117,974; 6,265,375, 6,355613, 6,407059, 6,512,092 and international applications WO 95/33765; WO 97/09344; WO 98/04583; WO 99/31121; WO 99/65508; WO 00/02898; WO 00/65467 and WO 02/062819.

As used herein “peptide” indicates a sequence of amino acids linked by peptide bonds. Functional derivatives of the peptides of the invention covers derivatives which may be prepared from the functional groups which occur as side chains on the residues or the N- or C-terminal groups, by means known in the art, and are included in the invention. These derivatives may, for example, include aliphatic esters of the carboxyl groups, amides of the carboxyl groups produced by reaction with ammonia or with primary or secondary amines, N-acyl derivatives of free amino groups of the amino acid residues formed by reaction with acyl moieties (e.g., alkanoyl or carbocyclic aroyl groups) or O-acyl derivatives of free hydroxyl groups (for example those of seryl or threonyl residues) formed by reaction with acyl moieties. Salts of the peptides of the invention contemplated by the invention are organic and inorganic salts.

The compounds herein disclosed may have asymmetric centers. All chiral, diastereomeric, and racemic forms are included in the present invention. Many geometric isomers of double bonds and the like can also be present in the compounds disclosed herein, and all such stable isomers are contemplated in the present invention.

The term “amino acid” refers to compounds, which have an amino group and a carboxylic acid group, preferably in a 1,2-1,3-, or 1,4-substitution pattern on a carbon backbone. α-Amino acids are most preferred, and include the 20 natural amino acids (which are L-amino acids except for glycine) which are found in proteins, the corresponding D-amino acids, the corresponding N-methyl amino acids, side chain modified amino acids, the biosynthetically available amino acids which are not found in proteins (e.g., 4-hydroxy-proline, 5-hydroxy-lysine, citrulline, ornithine, canavanine, djenkolic acid, β-cyanolanine), and synthetically derived α-amino acids, such as amino-isobutyric acid, norleucine, norvaline, homocysteine and homoserine. β-Alanine and γ-amino butyric acid are examples of 1,3 and 1,4-amino acids, respectively, and many others are well known to the art.

Some of the amino acids used in this invention are those which are available commercially or are available by routine synthetic methods. Certain residues may require special methods for incorporation into the peptide, and either sequential, divergent or convergent synthetic approaches to the peptide sequence are useful in this invention. Natural coded amino acids and their derivatives are represented by three-letter codes according to IUPAC conventions. When there is no indication, the L isomer was used. The D isomers are indicated by “D” or “(D)” before the residue abbreviation.

Conservative substitution of amino acids as known to those skilled in the art are within the scope of the present invention. Conservative amino acid substitutions includes replacement of one amino acid with another having the same type of functional group or side chain e.g. aliphatic, aromatic, positively charged, negatively charged. One of skill will recognize that individual substitutions, deletions or additions to peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art.

The following six groups each contain amino acids that are conservative substitutions for one another:

1) Alanine (A), Serine (S), Threonine (T);

2) Aspartic acid (D), Glutamic acid (E);

3) Asparagine (N), Glutamine (Q);

4) Arginine (R), Lysine (K), Histidine(H);

5) Isoleucine (I), Leucine (L), Methionine (M), Valine (V); and

6) Phenylalanine (F), Tyrosine (Y), Tryptophan (W).

“Permeability” refers to the ability of an agent or substance to penetrate, pervade, or diffuse through a barrier, membrane, or a skin layer. A “cell permeability moiety”, a “permeability enhancing moiety” or a “cell-penetration moiety” refers to any molecule known in the art which is able to facilitate or enhance penetration of molecules through membranes. Non-limitative examples include: hydrophobic moieties such as lipids, fatty acids, steroids and bulky aromatic or aliphatic compounds; hydrophilic moieties such as Arginine residues or guanidino-containing moieties; moieties which may have cell-membrane receptors or carriers, such as steroids, vitamins and sugars, natural and non-natural amino acids and transporter peptides.

### Pharmacology

The compounds of the present invention can be formulated into various pharmaceutical forms for purposes of administration. Pharmaceutical composition of interest may comprise at least one additive selected from a disintegrating agent, binder, flavoring agent, preservative, colorant and a mixture thereof, as detailed for example in “Handbook of Pharmaceutical Excipients”; Ed. A. H. Kibbe, 3rd Ed., American Pharmaceutical Association, USA.

For example, a compound of the invention, or its salt form or a stereochemically isomeric form, can be combined with a pharmaceutically acceptable carrier. Such a carrier can depend on the route of administration, such as oral, rectal, percutaneous or parenteral injection.

A “carrier” as used herein refers to a non-toxic solid, semisolid or liquid filler, diluent, vehicle, excipient, solubilizing agent, encapsulating material or formulation auxiliary of any conventional type, and encompasses all of the components of the composition other than the active pharmaceutical ingredient. The carrier may contain additional agents such as wetting or emulsifying agents, or pH buffering agents. Other materials such as anti-oxidants, humectants, viscosity stabilizers, and similar agents may be added as necessary.

For example, in preparing the compositions in oral dosage form, media such as water, glycols, oils, alcohols can be used in liquid preparations such as suspensions, syrups, elixirs, and solutions. Alternatively, solid carriers such as starches, sugars, kaolin, lubricants, binders, disintegrating agents can be used, for example, in powders, pills, capsules or tablets.

The pharmaceutically acceptable excipient(s) useful in the composition of the present invention are selected from but not limited to a group of excipients generally known to persons skilled in the art e.g. diluents such as lactose (Pharmatose DCL 21), starch, mannitol, sorbitol, dextrose, microcrystalline cellulose, dibasic calcium phosphate, sucrose-based diluents, confectioner's sugar, monobasic calcium sulfate monohydrate, calcium sulfate dihydrate, calcium lactate trihydrate, dextrates, inositol, hydrolyzed cereal solids, amylose, powdered cellulose, calcium carbonate, glycine, and bentonite; disintegrants; binders; fillers; bulking agent; organic acid(s); colorants; stabilizers; preservatives; lubricants; glidants/antiadherants; chelating agents; vehicles; bulking agents; stabilizers; preservatives; hydrophilic polymers; solubility enhancing agents such as glycerin, various grades of polyethylene oxides, transcutol and glycofiirol; tonicity adjusting agents; pH adjusting agents; antioxidants; osmotic agents; chelating agents; viscosifying agents; wetting agents; emulsifying agents; acids; sugar alcohol; reducing sugars; non-reducing sugars and the like, used either alone or in combination thereof. The disintegrants useful in the present invention include but not limited to starch or its derivatives, partially pregelatinized maize starch (Starch 1500®), croscarmellose sodium, sodium starch glycollate, clays, celluloses, alginates, pregelatinized corn starch, crospovidone, gums and the like used either alone or in combination thereof. The lubricants useful in the present invention include but not limited to talc, magnesium stearate, calcium stearate, sodium stearate, stearic acid, hydrogenated vegetable oil, glyceryl behenate, glyceryl behapate, waxes, Stearowet, boric acid, sodium benzoate, sodium acetate, sodium chloride, DL-leucine, polyethylene glycols, sodium oleate, sodium lauryl sulfate, magnesium lauryl sulfate and the like used either alone or in combination thereof. The anti-adherents or glidants useful in the present invention are selected from but not limited to a group comprising talc, corn starch, DL-leucine, sodium lauryl sulfate, and magnesium, calcium and sodium stearates, and the like or mixtures thereof. In another embodiment of the present invention, the compositions may additionally comprise an antimicrobial preservative such as benzyl alcohol. In an embodiment of the present invention, the composition may additionally comprise a conventionally known antioxidant such as ascorbyl palmitate, butylhydroxyanisole, butylhydroxytoluene, propyl gallate and/or tocopherol. In another embodiment, the dosage form of the present invention additionally comprises at least one wetting agent(s) such as a surfactant selected from a group comprising anionic surfactants, cationic surfactants, non-ionic surfactants, zwitterionic surfactants, or mixtures thereof. The wetting agents are selected from but not limited to a group comprising oleic acid, glyceryl monostearate, sorbitan monooleate, sorbitan monolaurate, triethanolamine oleate, polyoxyethylene sorbitan monooleate, polyoxyethylene sorbitan monolaurate, sodium oleate, sodium lauryl sulfate and the like, or mixtures thereof. In yet another embodiment, the dosage form of the present invention additionally comprises at least one complexing agent such as cyclodextrin selected from a group comprising but not limited to alpha-cyclodextrin, beta-cyclodextrin, betahydroxy-cyclodextrin, gamma-cyclodextrin, and hydroxypropyl beta-cyclodextrin, or the like. In yet another embodiment, the dosage form of the present invention additionally comprises of lipid(s) selected from but not limited to glyceryl behenate such as Compritol® ATO888, Compritol® ATO 5, and the like; hydrogenated vegetable oil such as hydrogenated castor oil e.g. Lubritab®; glyceryl palmitostearate such as Precirol® ATO 5 and the like, or mixtures thereof used either alone or in combination thereof. It will be appreciated that any given excipient may serve more than one function in the compositions according to the present invention.

For parenteral compositions, the carrier can comprise sterile water. Other ingredients may be included to aid in solubility. Injectable solutions can be prepared where the carrier includes a saline solution, glucose solution or mixture of both.

Injectable suspensions can also be prepared. In addition, solid preparations that are converted to liquid form shortly before use can be made. For percutaneous administration, the carrier can include a penetration enhancing agent or a wetting agent.

It can be advantageous to formulate the compositions of the invention in dosage unit form for ease of administration and uniformity of dosage. “Dosage unit form” refers to physically discrete units suitable as unitary dosages, each unit containing a pre-determined quantity of active ingredient calculated to produce the desired therapeutic effect in association with the chosen carrier.

Apart from other considerations, the fact that the novel active ingredients of the invention are peptides, peptide analogs or peptidomimetics, dictates that the formulation be suitable for delivery of these types of compounds. Although in general peptides are less suitable for oral administration due to susceptibility to digestion by gastric acids or intestinal enzymes. According to the present invention, novel methods of backbone cyclization are being used, in order to synthesize metabolically stable and oral bioavailable peptidomimetic analogs. The preferred route of administration of peptides of the invention is oral administration.

Other routes of administration are intra-articular, intravenous, intramuscular, subcutaneous, intradermal, or intrathecal.

Pharmaceutical compositions of the present invention may be manufactured by processes well known in the art, e.g., by means of conventional mixing, dissolving, granulating, grinding, pulverizing, dragee-making, levigating, emulsifying, encapsulating, entrapping or lyophilizing processes.

Pharmaceutical compositions, which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added.

For injection, the compounds of the invention may be formulated in aqueous solutions, preferably in physiologically compatible buffers such as Hank's solution, Ringer's solution, or physiological saline buffer. For transmucosal administration, penetrants appropriate to the barrier to be permeated are used in the formulation. Such penetrants for example polyethylene glycol are generally known in the art.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

For buccal administration, the compositions may take the form of tablets or lozenges formulated in conventional manner.

For administration by inhalation, the variants for use according to the present invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in an inhaler or insufflator may be formulated containing a powder mix of the peptide and a suitable powder base such as lactose or starch.

Pharmaceutical compositions for parenteral administration include aqueous solutions of the active ingredients in water-soluble form. Additionally, suspensions of the active compounds may be prepared as appropriate oily injection suspensions. Suitable natural or synthetic carriers are well known in the art (Pillai et al., 2001, Curr. Opin. Chem. Biol. 5, 447). Optionally, the suspension may also contain suitable stabilizers or agents, which increase the solubility of the compounds, to allow for the preparation of highly concentrated solutions. Alternatively, the active ingredient may be in powder form for reconstitution with a suitable vehicle, e.g., sterile, pyrogen-free water, before use.

The compounds of the present invention may also be formulated in rectal compositions such as suppositories or retention enemas, using, e.g., conventional suppository bases such as cocoa butter or other glycerides.

Pharmaceutical compositions suitable for use in context of the present invention include compositions wherein the active ingredients are contained in an amount effective to achieve the intended purpose. More specifically, a therapeutically effective amount means an amount of a compound effective to prevent, alleviate or ameliorate symptoms of a disease of the subject being treated. Determination of a therapeutically effective amount is well within the capability of those skilled in the art.

Toxicity and therapeutic efficacy of the peptides described herein can be determined by standard pharmaceutical procedures in cell cultures or experimental animals, e.g., by determining the IC50 (the concentration which provides 50% inhibition) and the LD50 (lethal dose causing death in 50% of the tested animals) for a subject compound. The data obtained from these cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition (e.g. Fingl, et al., 1975, in “The Pharmacological Basis of Therapeutics”, Ch. 1 p. 1).

Those skilled in the art of treatment of MS can determine the effective daily amount. Generally, an effective amount can be from 0.01 mg/kg to 50 mg/kg body weight and, more preferably, from 0.1 mg/kg to 10 mg/kg body weight

The precise dosage and frequency of administration depends on the particular compound of the invention being used, as well as the particular condition being treated, the severity of the condition, the age, weight, and general physical condition of the subject being treated, as well as other medication being taken by the subject, as is well known to those skilled in the art. It is also known that the effective daily amount can be lowered or increased depending on the response of the subject or the evaluation of the prescribing physician. Thus, the ranges mentioned above are only guidelines and are not intended to limit the scope of the use of the invention.

The combination of a compound of the invention with another agent used for treatment of MS can be used. Such combination can be used simultaneously, sequentially or separately. Such agents may include, for example, glucocorticoids, immunomodulatory drugs such as interferon beta, glatiramer acetate, fingolimod and mitoxanthrone.

The following examples are presented in order to more fully illustrate some embodiments of the invention. They should, in no way be construed, however, as limiting the broad scope of the invention. One skilled in the art can readily devise many variations and modifications of the principles disclosed herein without departing from the scope of the invention.

### General Procedures

### Chemistry General

All starting materials were purchased from commercial sources and were used without further purification. Nuclear magnetic resonance (NMR) spectra during synthesis were recorded on a Bruker AMX 300, Bruker 400 or Bruker 500 MHz spectrometer. Chemical shifts are reported downfield, relative to internal solvent peaks. Coupling constants J are reported in Hz. High Resolution Mass spectrometry (HRMS) spectra were recorded on nanospray ionization LTQ orbitrap. Matrix assisted laser desorption ionization (MALDI)-time of flight (TOF) (MALDI-TOF) Mass spectra were recorded on a PerSeptive Biosystems MALDI-TOF MS, using -cyano-4-hydroxycinnamic acid as matrix. Thin layer chromatography (TLC) was performed on Merck aluminum sheets silica gel 60 F254. Column chromatography was performed on Merck silica gel 60 (230-400 mesh).

Peptides purity was determined by analytical HPLC, peptides below 95% purity were excluded from further examination (see supporting information). Analytical HPLC was performed on Vydac analytical columns (C18, 5 4.6 mm×250 mm (218TP54)) using Merck-Hitachi system: Model LaChrom with a L-7100 pump, L-7200 autosampler, L-7400 UV/Vis detector and a D-7000 interface. Products were assayed at 215 and 220 nm. The mobile phase consisted of a gradient system, with solvent A corresponding to TDW with 0.1% TFA and solvent B corresponding to acetonitrile (ACN) with 0.1% TFA. The mobile phase started with 95% A from 0 to 5 min followed by a linear gradient from 5% B to 95% B from 5 to 55 min. The gradient remained at 95% B for an additional 5 min and then was reduced to 95% A and 5% B from 60 to 65 min. The gradient remained at 95% A for additional 5 min to achieve column equilibration. The flow rate of the mobile phase was 1 mL/min. Peptide purification was performed by reversed phase semi-preparative HPLC on a Merck-Hitachi 665A model equipped with a preparative pump (30 ml/min) and a high flow UV/Vis detector using semipreparative Vydac column (C18, 5, 10×250 (208TP510)) flow rate of the mobile phase was 4.5 mL/min. All semi preparative HPLC runs were carried out using a gradient system similar to the one used in for the analytical HPLC.

Analytical RP-HPLC were recorded at 220 nm at a flow of 1 ml/min on Merck-Hitachi system (LaChrom with a L-7100 pump, L-7200 autosampler, L-7400 UV/Vis detector and a D-7000 interface) on Phenomenex RP-18 column (C18, 5i, 4.6×75 mm (Luna)). Using the same solvent system previously described, the mobile phase started with 95% A from 0 to 5 min followed by a linear gradient from 5% B to 95% B from 5 to 17 min. The gradient remained at 95% B for an additional 4 min and then was reduced to 95% A from 21 to 25 min. The gradient remained at 95% A for additional 5 min to achieve column equilibration. Semi-preparative HPLC were recorded at 220 nm on Phenomenex RP-18 column (C18, 10 250×10 mm, 110 Å (Gemini)). Using the same solvent system previously described, the mobile phase started with 95% A from 0 to 5 min followed by a linear gradient from 5% B to 35% B from 5 to 30 min, then to 95% B in 15 min, the gradient remained at 95% B for an additional 5 min and then was reduced to 95% A in 10 min. The gradient remained at 95% A for additional 5 min to achieve column equilibration.

### Circular Dichroism (CD)

CD spectra of the peptides were recorded on a JASCO J-810 Spectrophotometer (JASCO, Japan) using the supplied Spectra-Manager software. The temperature was kept constant at 25° C. using a temperature controlled water bath. Samples were made fresh from stock before each measurement. Peptides were dissolved in 2,2,2-trifluorethanol (TFE) and diluted with water to give 200 μM concentration. Spectra were recorded in the wavelength range I=195-260 nm, with 5 accumulations for each measurement and a data pitch of 0.1 nm using 0.1 cm quartz cells (Sterna, Calif.). Background CD spectra were recorded and subtracted from each spectrum.

### Functional Inhibitory Potency Determination by In Vitro Methods

The peptides are tested by using a lymphocyte culture system as described for example in McCarthy M, deVellis J (1980). J Cell Biol 85: 890-902. Briefly, lymphocytes obtained from animals immunized with MOG protein (one of the major myelin proteins) for EAE induction, are cultured and stimulated with myelin peptides or non-specific mitogens, in the presence or absence of various concentrations of the tested peptides. The proliferation of the lymphocytes and the production of inflammatory cytokines is evaluated by ELISA methods and by thymidine incorporation assays.

### Assessment of Intestinal Absorption Properties

Transport studies are performed through the Caco-2 monolayer mounted in an Using-type chamber set-up with continuous transepithelial electrical resistance (TEER) measurements to assure TEER between 800 and 1200 Ω*cm2. HBSS supplemented with 10 mM MES and adjusted to pH 6.5 will be used as transport medium in the donor compartment and pH 7.4 in the acceptor compartment. The donor solution contains the test compound. The effective permeability coefficient is calculated from concentration-time profiles of each of the tested compounds in the acceptor chamber.

### Metabolic Stability

The enzymatic reaction is performed similar to what described in Ovadia et al. (2009, Bioorg Med Chem 18, 580-589): 2 mM stock solutions of the tested compounds are diluted with serum or purified brush border membrane vesicles (BBMVs) solution to a final concentration of 0.5 mM. During incubation at 37° C. samples are taken for a period of 90 minutes. The enzymatic reaction is stopped by adding 1:1 v/v of ice cold acetonitrile and centrifuge (4000 g, 10 min) before analysis.

The BBMVs are prepared from combined duodenum, jejunum, and upper ileum (male Wistar rats) by a Ca++ precipitation method (Gante, J., Angew Chem Int Edit 1994, 33, (17), 1699-1720; Hess et al., ibid). Purification of the BBMVs is assayed using GGT, LAP and alkaline phosphatase as membrane enzyme markers.

### Pharmacokinetic (PK) Studies

The PK studies are performed in conscious Wistar male rats. An indwelling cannula is implanted in the jugular vein 24 hr before the PK experiment to allow full recovery of the animals from the surgical procedure. Animals (n=5) receive either an iv bolus dose or oral dose of the investigated compound. Blood samples (with heparin, 15 U/ml) are collected at several time points for up to 24 hrs post administration and assayed by HPLC-MS method. Noncompartmental pharmacokinetic analysis is performed using WinNonlin software.

### In Vivo Studies

Effective peptides are used to treat mice with EAE (the animal model of MS) as described for example in Owens T. and Sriram S, Neurologic Clinics (1995) 13(1):51-73. Specifically, C57BI mice are immunized with the MOG protein in adjuvant and the paralysis disease which appears 10-14 days following the induction, and is evaluated daily. Two groups of animals are treated with two doses of the peptide administered orally by cannula on a daily basis, from the day of EAE-induction. One month after the disease onset, the animals are sacrificed and their brains and spinal cords are processed for histopathological analysis (performed by a blinded for the treatment arm, neuropathologist). This includes the evaluation of the number of inflammatory infiltrates and the number of cells per infiltrate, the degree of demyelination and of axonal damage.

### EXAMPLES

### Example 1

### Determining the Active Site of CCR2 Dimerization by Linear Peptides

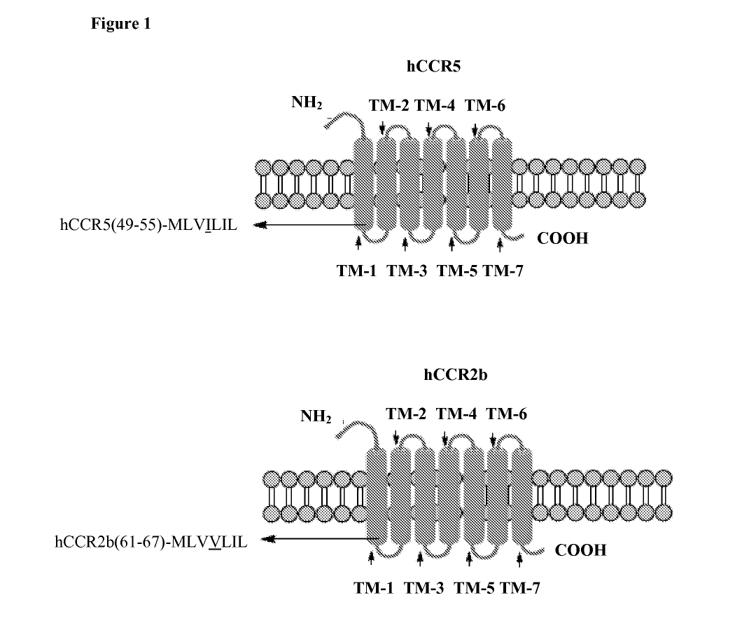

Chemokine receptors are highly homologous although they participate in different mechanisms and signal transduction pathways. Several segments of the helix bundle of chemokine receptors take part in dimerization in response to chemokine binding. Linear peptides derived from the putative dimerization regions proved to bind the chain association and, as a result, inhibited the chemokine—induced cell migration. CCR2 dimerization site is only partially resolved and not all of the pharmacophores involved in the protein-protein interactions have been identified. Based on homology to CCR5, the first transmembrane segment of CCR2 (TM-1, FIG. 1) was chosen for design of inhibitory molecules. A short heptapeptide derived from the chemokine receptor hCCR2b (residues 61-67) was synthesized having the sequence MLVVLIL (SEQ ID NO: 2). This heptapeptide has a unique hydrophobic sequence that includes two valines, three leucines and one isoleucine. It is almost identical to the dimerization region of CCR5 but differs in one amino acid (FIG. 1).

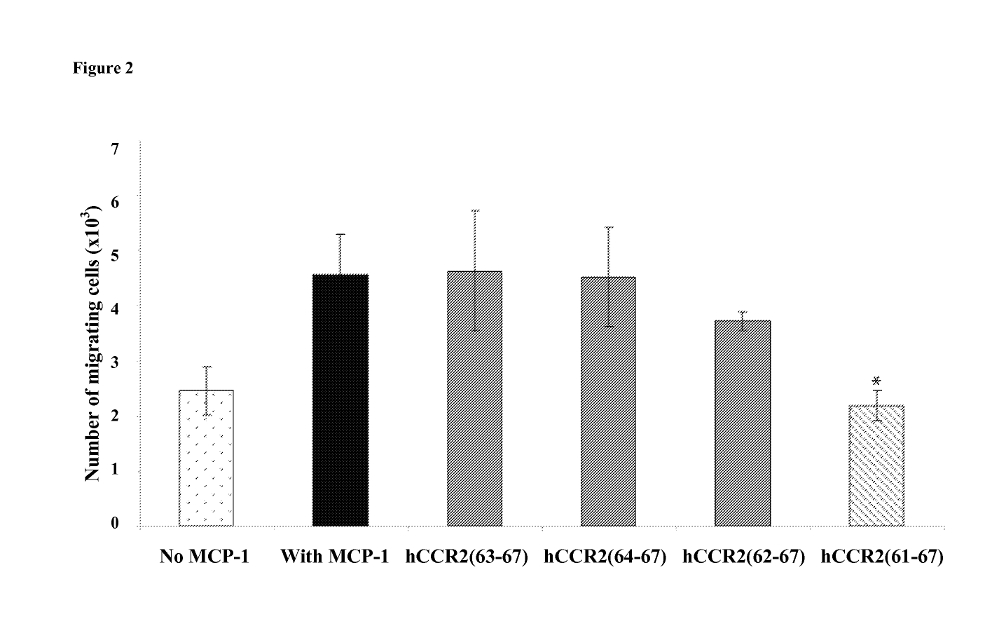

A transwell migration assay (Bignold, L. P., J Immunol Methods 1987, 105, (2), 275-80) was performed using MCP-1 as a chemoattractant. The chemokine MCP-1 reacts only with the chemokine receptors CCR2 and CCR4. Human acute monocyte leukemia cell line (THP-1) was selected for this study since it does not express CCR4 (Imai et al., J Biol Chem 1997, 272, (23), 15036-42), hence, MCP-1 chemotactic effects can be attributed solely to the specific CCR2/MCP-1 interaction. In this experiment, THP-1 cells were placed in the upper well of the trans-migration plate and specific migration was induced by placing MCP-1 in the lower well. The cells were allowed to migrate spontaneously or toward the chemokine, and counted after migration. To evaluate the effect of hCCR2b(61-67) on MCP-1-induced migration, the peptide (10 μM) was incubated with the cells and the number of migrating cells in the lower wells were determined. The results show that the number of migrating cells in monocytes treated with the hCCR2b(61-67) peptide was reduced compared with the untreated control (FIG. 2 striped) thus indicating that the heptapeptide hCCR2b(61-67) inhibits MCP-1-mediated migration.

The inhibitory effect of the hCCR2b(61-67) heptapeptide, lead to synthesis of a series of truncated hCCR2b(61-67) derivatives in search for smaller active peptides. Further tests of the shorter peptides demonstrated that they fail to inhibit MCP-1-mediated migration (FIG. 2 gray). It was therefore concluded that the heptapeptide is the shortest peptide able to bind to the receptor and prevent CCR2 dimerization. A new helix stabilization method was developed and used to construct a peptide mimetic capable of inhibiting the CCR2 dimerization.

### Example 2

### Urea Backbone Cyclic Helix Mimetics

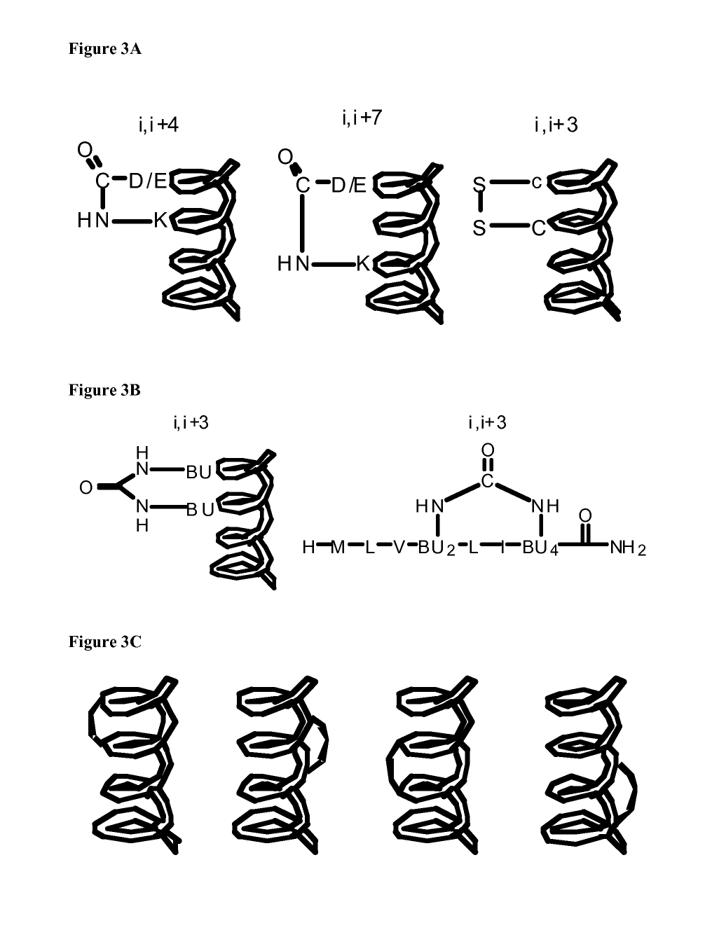

Stabilization of putative helices might lead to a better understanding of the secondary structure and facilitate rational drug design. The general structure of an alpha helix is well characterized and in most cases consists of i,i+4 hydrogen bonds. However, i,i+3 (310 helix) and i,i+5 (π helix) hydrogen bonds can also be found in other helical structures. The specific helix structure determines the function of the segment and controls its orientation and interactions. Generally, helices are stabilized by covalently connecting either positions i,i+4, i,i+7 and in some cases i,i+3 (FIG. 3A). Although many novel methods have been reported for helix mimetics, amide bonds connecting Asp/Glu to Lys are the most frequently used for cyclization. Several studies have shown that the size of the cyclic ring, along with the type of ring chemistry and the position of the anchor, influence the helical nature of the peptide. The importance of helix mimetics to drug development is immense, and there is a strong demand for rational conversion of helices in drug-like molecules.

Urea backbone cyclization was herein used to perform ring a position scan since it complies with the above demands. In urea BC, two Alloc protected Glycine Building Units (AGBU) are incorporated to the peptide and later connected by a urea bond on solid support to form a ring (Hurevich et al., J Pept Sci 2010, 16, (4), 178-185). Urea BC ring position scanning is a method in which the position of anchoring the AGBU is changed in each peptide (FIG. 3C). For the current study, a systematic replacement of two of amino acids in hCCR2b(61-67) by AGBU was performed (i to i+3, i+1 to i+4, i+2 to i+5, etc.). By keeping a constant distance of two amino acids between the AGBU, a i,i+3 helix mimetic cyclization scan was performed. Helix stabilization of hCCR2b(61-67) was screened for by replacing the amino acid with an AGBU bearing an alkyl chain with n=2 and n=4 instead of the i+3 position (FIG. 3B).

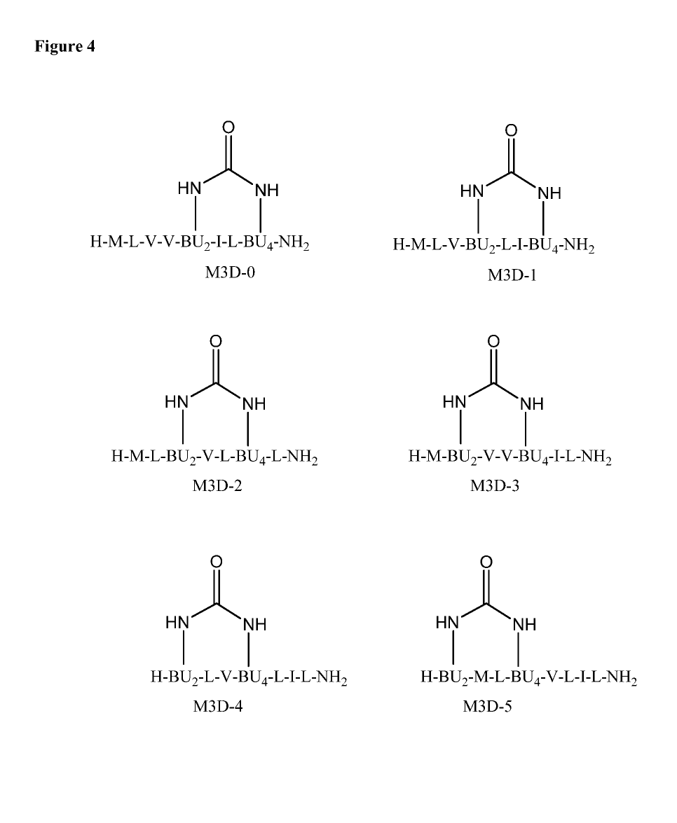

A series of five hCCR2b(61-67) urea BC peptides (M3D-m library, FIG. 4) was synthesized. Out of the five peptide mimetics, one (M3D-2) failed to close and was unavailable for further examination.

### Example 3

### Microwave Assisted Synthesis of M3D-1

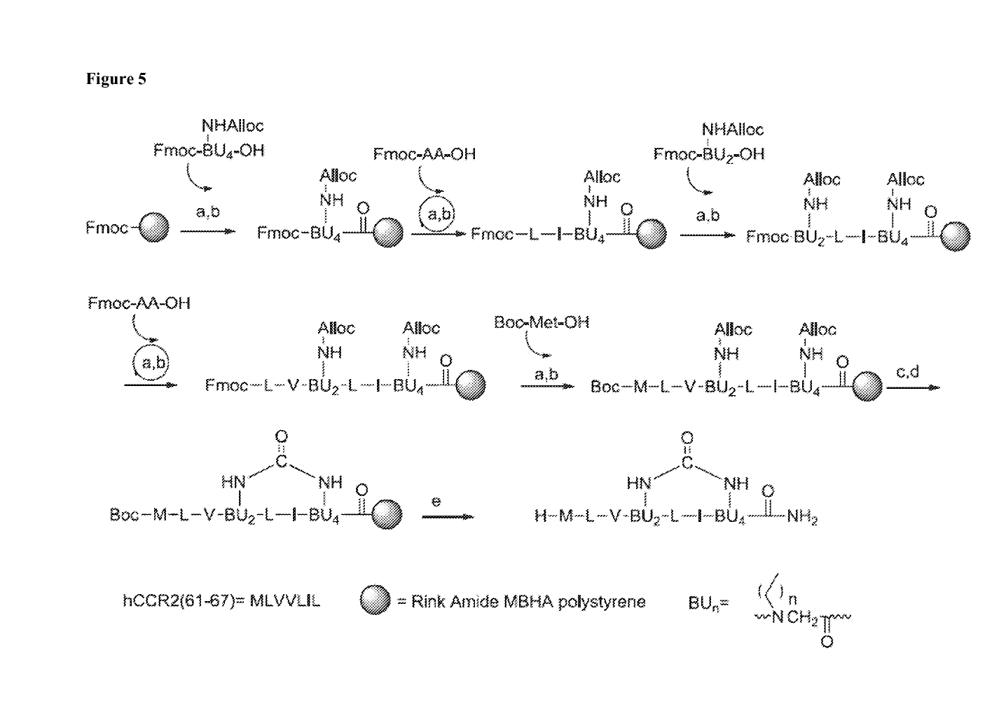

M3D-1 was prepared by synthesizing and using two non-natural building blocks (Hurevich et al. ibid) of Alloc protected Glycine Building Units (AGBU) as described in FIG. 5. Microwave assisted peptide synthesis (MAPS) was used to overcome synthetic limitations encountered in the early stages of the synthesis. [2-(7-Aza-1H-benzotriazole-1-yl)-1,1,3,3-tetramethyluronium hexafluorophosphate] (HATU) was used instead of [2-(1H-benzotriazole-1-yl)-1,1,3,3-tetramethyluronium hexafluorophosphate] (HBTU) to surmount coupling difficulties during the synthesis of the linear precursor. The precyclic precursor was assembled by repeating a fast cycle of coupling and deprotection. A typical amino acid coupling cycle included a coupling step (5 min), two washes (4 min), 9-fluorenylmethyloxycarbonyl (Fmoc) removal (6 min) and two washes (5 min). A tbutoxycarbonyl (Boc) protecting group was used to protect the amino terminus to avoid undesired Fmoc removal during the Alloc removal step. After assembling the linear precursor, the allyloxycarbonyl (Alloc) groups were removed by using an efficient methodology involving PhSiH3 as scavenger (Bleul, et al., Nature 1996, 382, (6594), 829-33). The free amino groups on the N-alkyl chain of the glycine derivatives were bonded using a bis(trichloromethyl)carbonate (BTC)-mediated urea cyclization procedure (Hurevich et al., Heterocycles 2007, 73, (1), 617-625). At the last step, a special cleavage mixture containing 1,2-ethanedithiol (EDT) was used to overcome methionine oxidation during cleavage. Coupling using HATU enabled the use of the same method for all amino acids including coupling to the AGBU. This methodology has an advantage over procedures previously used for the preparation of BC peptides since a complete assembly of one BC peptide is much faster than by other methods (Barda et al., Nucl Med Biol 2004, 31, (7), 921-33; Qvit et al., Biopolymers 2009, 91, (2), 157-168).

The synthesis of large quantities of M3D-1 is performed by MW assisted SPPS according to the scale up procedures described above. The peptide is purified by HPLC and characterized by MS and Circular dichroism (CD).

### Example 4

### Structural Screening

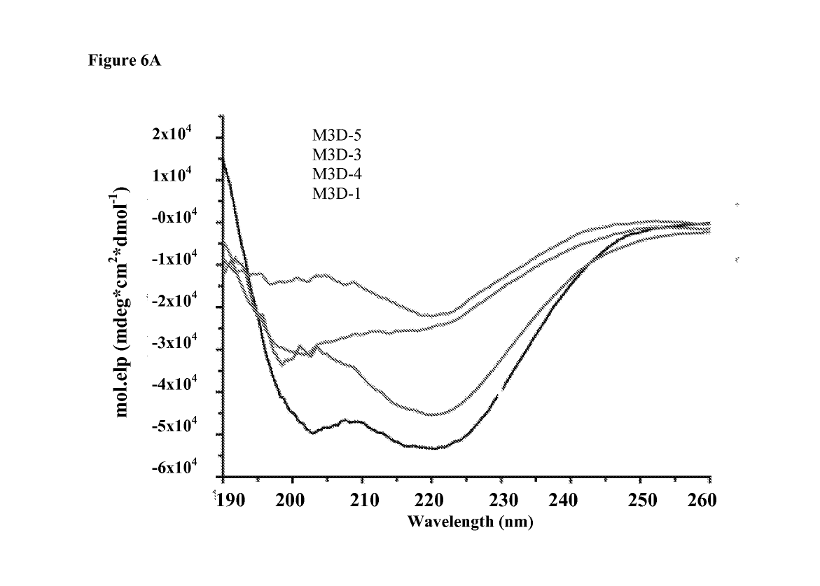

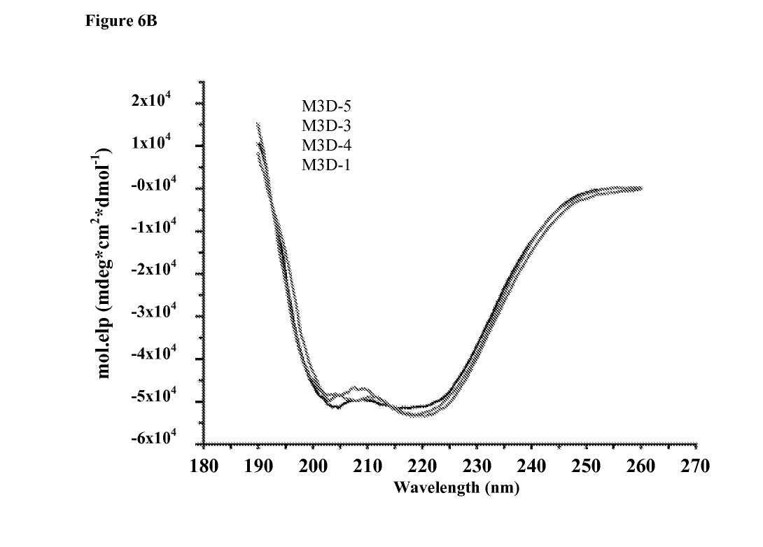

A CD screening of the urea BC peptides was performed in order to test which urea BC peptide analog of hCCR2b(61-67) stabilize a helical structure and is a potential CCR2 dimerization blocker. To compare the linear peptide hCCR2b(61-67), all CD measurements were performed in a 10% TFE solution in water. Results (FIG. 6A) clearly suggest that some analogs induce the desired secondary helical structure (compounds M3D-1 and M3D-4). In particular, the cyclic peptide M3D-1 showed a typical helix CD spectrum. The CD results clearly indicated a helix structure as can be seen by the distinctive minima around 205 and 220 nm (FIGS. 6A and B). The effect of TFE on M3D-1 CD spectra was evaluated by changing the ratio of TFE/water (FIG. 6B). Only a minor effect was observed with a higher percentage of TFE, leading to the conclusion that the TFE is not responsible for the structural stability of M3D-1. When compared to the linear parent peptide it is clear that the cyclic peptide M3D-1 achieved helix stabilization.

### Example 5

### Stability of MD-3

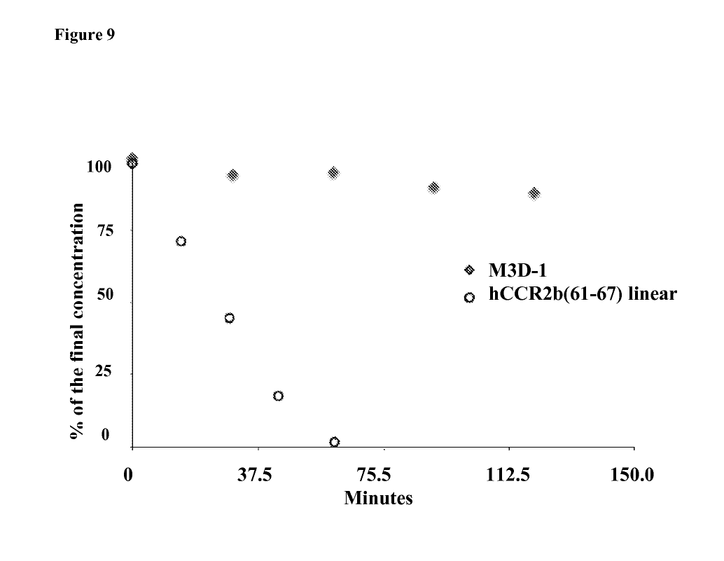

M3D-1 was tested for metabolic stability to degradation by intestinal peptidases using the BBMV assay described above. The linear CCR2b(61-67) peptide was used as a control. As can be seen in FIG. 9, M3D-1 was stable for at least 3 hours while the linear peptide completed degraded after about one hour.

### Example 6

### Biological Evaluation of MD-3

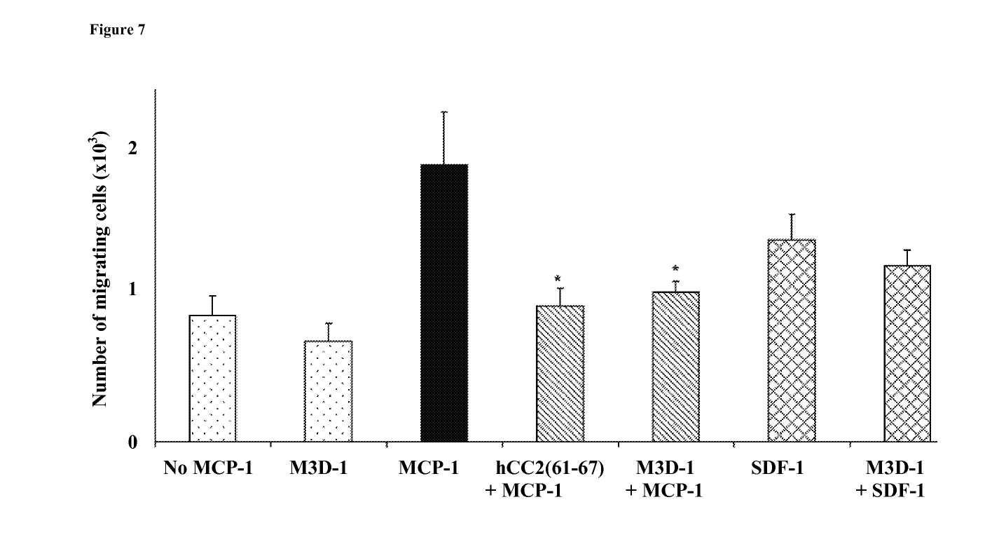

The simple transwell migration assay (Mandelboim, O. Protocol Exchange (2006) doi:10.1038/nprot.2006.210) was used for evaluating the chemotactic migration in the presence of M3D-1. M3D-1 inhibited THP-1 cells migration towards MCP-1 (FIG. 7 stripes). The similar inhibitory activity of M3D-1 to that of hCCR2(61-67) indicates that M3D-1 acquired the bioactive conformation. In vitro cytotoxicity assay showed that M3D-1 is not toxic to cells and thus demonstrate that the effect is solely related to cells migration inhibition.

To prove that the cyclic peptides interfere only with MCP-1 mediated migration, the effect of M3D-1 on stromal cell-derived factor-1 (SDF-1 or CXCL12) induced migration was determined. SDF-1 is known to have strong chemotactic effects following specific interaction with the receptor CXCR4 (Bleul et al., ibid). It is shown that no significant inhibition by M3D-1 was detected in cells migrating towards SDF-1 (FIG. 7 crosshatch). The results indicate that M3D-1 is specific receptor inhibitor lacking cross reactivity with other chemokine receptors.

To confirm that M3D-1 does not target spontaneous migration of monocytes, a second control assay was performed. THP-1 cells spontaneous migration was evaluated without additional chemokines. THP-1 cells were allowed to migrate with or without M3D-1 addition (FIG. 7 dots) and the results indicate that in the absence of chemokine, there is no significant difference in the number of cells that migrate.

These results suggest that M3D-1 inhibits the chemokine—induced migration and not the spontaneous cell movement. The biological data indicate that M3D-1 interferes in a specific manner with the signal transduction pathway resulting from MCP-1/CCR2 interaction and consequently blocks the chemokine mediated cell migration.

### Example 7

### Design, Synthesis and Screening of Bridge Chemistry and Bridge Position Libraries Based on M3D-1

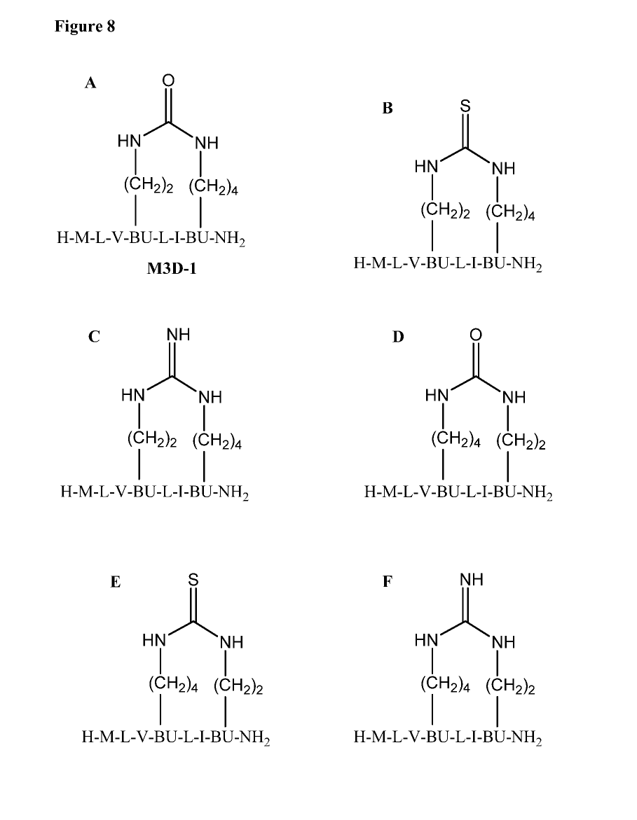

In order to improve the PK and PD of M3D-1 two focused libraries based on M3D-1 (compound A in FIG. 8) were prepared. The first library which is comprised of two analogs (compounds B and C in FIG. 8) include analogs of M3D-1 in which the bridge position and ring size is kept the same as in M3D-1 but the bridge chemistry is changed from urea bridge into thiourea and guanidine bridges. In compound D, the building units forming the bridge were interchanged.

Analogs C and F are designed to modify the hydrophobic character of the M3D-1 compound. In compounds E and F both bridge position (building units) and bridge chemistry modifications were incorporated.

The peptides from the various ring size library are initially screened by comparing their CD spectra to that of the linear parent peptide on one hand and the known spectra of alpha helix on the other. The peptides are then be screened by testing their efficacy in suppressing the clinical and histopathological manifestations of the animal model of MS, EAE.

### Example 8

### Design, Synthesis and Screening of M3D-1 Analogs Comprising Hydrophilic Moiety

To increase hydrophilicity of the M3D-1 peptide, analogs comprising hydrophilic moieties were designed and synthesized. The hydrophilic moiety is attached to the amino terminus of the peptide and/or inserted as part of the bridge. In addition, bridge size is modified by using different number of methylene groups in each building unit as described in formula I:

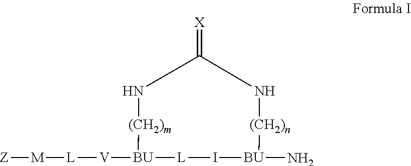

wherein m is an integer of 2-6; n is an integer of 2-6; X is selected from the group consisting of: O, S and NH; Z is a cell permeability moiety such as an hydrophilic moiety or triglycerol; and BU designates a N^{α}-ω-functionalized amino acid residue. Some of the analogs synthesized are:

M3D-1 GB comprising a guanidino bridge:

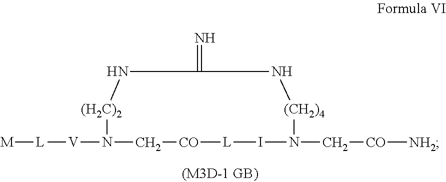

M3D-1 2G comprising a guanidino bridge and a guanidino N-terminus:

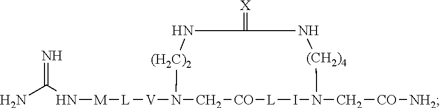

M3D-1 3G comprising a guanidino bridge, a arginine residue and a guanidino amino terminus:

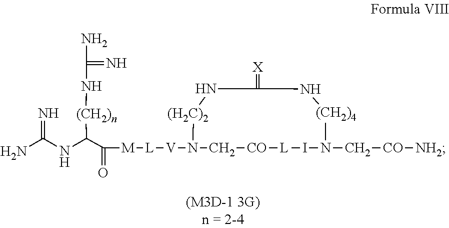

M3D-1 R series comprising 1-3 Arginyl residues to the amino terminus:

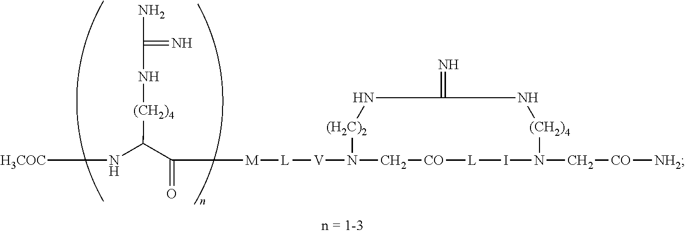

M3D-1 HP comprising a guanidino bridge and a triglycerol residue (1,3-Bis(2,3-dihydroxypropyl)-2-propane carboxylic acid) attached to the amino terminus:

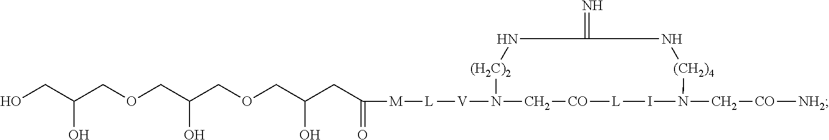

M3D-1 Glu comprising a guanidino bridge and a glucose residue attached to the amino terminus:

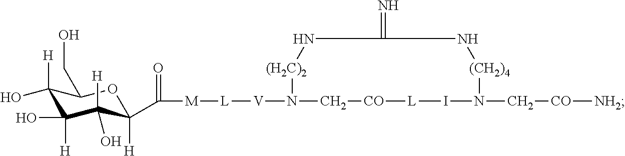

Trehalosyl-M3D-1 comprising a trehalose attached to the amino terminus:

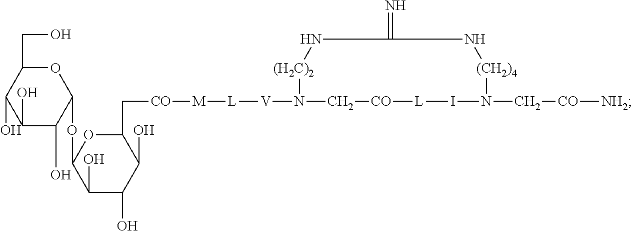

PEGylated M3D-1 comprising polyethylenglycol (PEG) attached to the amino terminus:

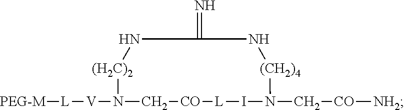

M3D-1 with bridge chemistries and guanidino alpha amine modification (X is O, N or S):

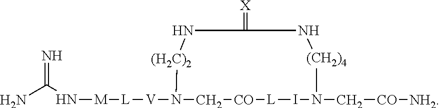

The compounds are tested for their helical structure and for their permeability and activity as described above.

While the present invention has been particularly described, persons skilled in the art will appreciate that many variations and modifications can be made. Therefore, the invention is not to be construed as restricted to the particularly described embodiments, and the scope and concept of the invention will be more readily understood by reference to the claims, which follow.

1. A synthetic peptide of 5-15 amino acid residues comprising a sequence derived from the sequence of transmembrane 1 (TM-1) of the Chemokine (C-C motif) receptor 2 (CCR2), wherein the peptide structure is stabilized by covalently connecting at least one N^{α}-ω-functionalized derivative of an amino acid residue added to the sequence, or substituted for an amino acid residue in the sequence, with a moiety selected from the group consisting of: another N^{α}-ω-functionalized derivative of an amino acid residue; the side chain of an amino acid in the peptide sequence; or one of the peptide terminals, to form a backbone cyclized helical peptide.

2. The synthetic peptide of claim 1 wherein the CCR2 receptor is human CCR2b subtype (SEQ ID NO: 1).

3. The synthetic peptide of claim 2 wherein the sequence derived from TM-1 comprises at least five amino acids of the sequence MLVVLIL (SEQ ID NO: 2), corresponding to amino acid residues 61-67 of human CCR2b.

4. The synthetic peptide of claim 3 comprising the sequence MLVVLIL (SEQ ID NO: 2) wherein two amino acid residues were substituted with N^{α}-ω-functionalized derivatives of amino acid residues connected to form backbone cyclization.

5. The synthetic peptide of claim 4 wherein the Valine (V) residue at position 4 of SEQ ID NO: 2 is replaced with a N^{α}-ω-functionalized amino acid residue.

6. The synthetic peptide of claim 4 wherein backbone cyclization is between positions selected from the group consisting of: 4-7, 1-4, 4 to C-terminus, and 4 to N-terminus.

7. The synthetic peptide of claim 1 wherein a covalent bond used for connecting the at least one N^{α}-ω-functionalized amino acid residue is selected from the group consisting of: amide bond, disulfide bond, and urea bond.

8. The synthetic peptide of claim 1 wherein the peptide consists of 7-12 amino acid residues.

9. The synthetic peptide of claim 1 further comprising a permeability enhancing moiety, conjugated to the peptide.

10. The synthetic peptide of claim 1 represented by Formula I: 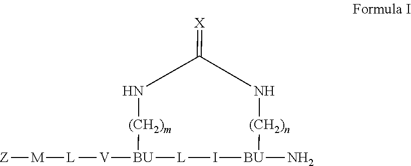
wherein m is an integer of 2-6; n is an integer of 2-6; X is selected from the group consisting of: O, S and NH; Z is selected from the group consisting of: hydrogen, a carbohydrate moiety, a hydrophilic moiety, a polyethylene glycol (PEG), and a triglycerol; and wherein BU designates a N^{α}-ω-functionalized amino acid residue.

11. The synthetic peptide according to claim 10 wherein m is 2 and n is 4 or wherein m is 4 and n is 2.

12. The synthetic peptide according to claim 10 wherein BU designates a N^{α}-ω-functionalized Glycine residue.

13. The synthetic peptide according to claim 10 wherein BU designates a N^{α}-ω-functionalized residue of a natural or synthetic amino acid other than glycine.

14. The synthetic peptide according to claim 10 wherein Z is selected from the group consisting of: 1-5 hydrophilic amino acid residues, a guanidino group, a carbohydrate moiety and a moiety comprising one to three Arginine residues.

15. The synthetic peptide according to claim 14 wherein the carbohydrate moiety is a glucose or trehalose residue or a derivative thereof.

16. The synthetic peptide of claim 1, selected from the group consisting of: 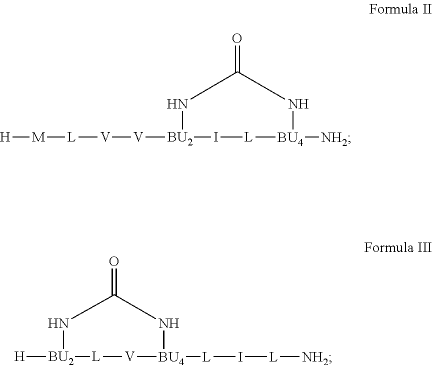
wherein, BU designates a N^{α}-ω-functionalized amino acid residue of the formula:

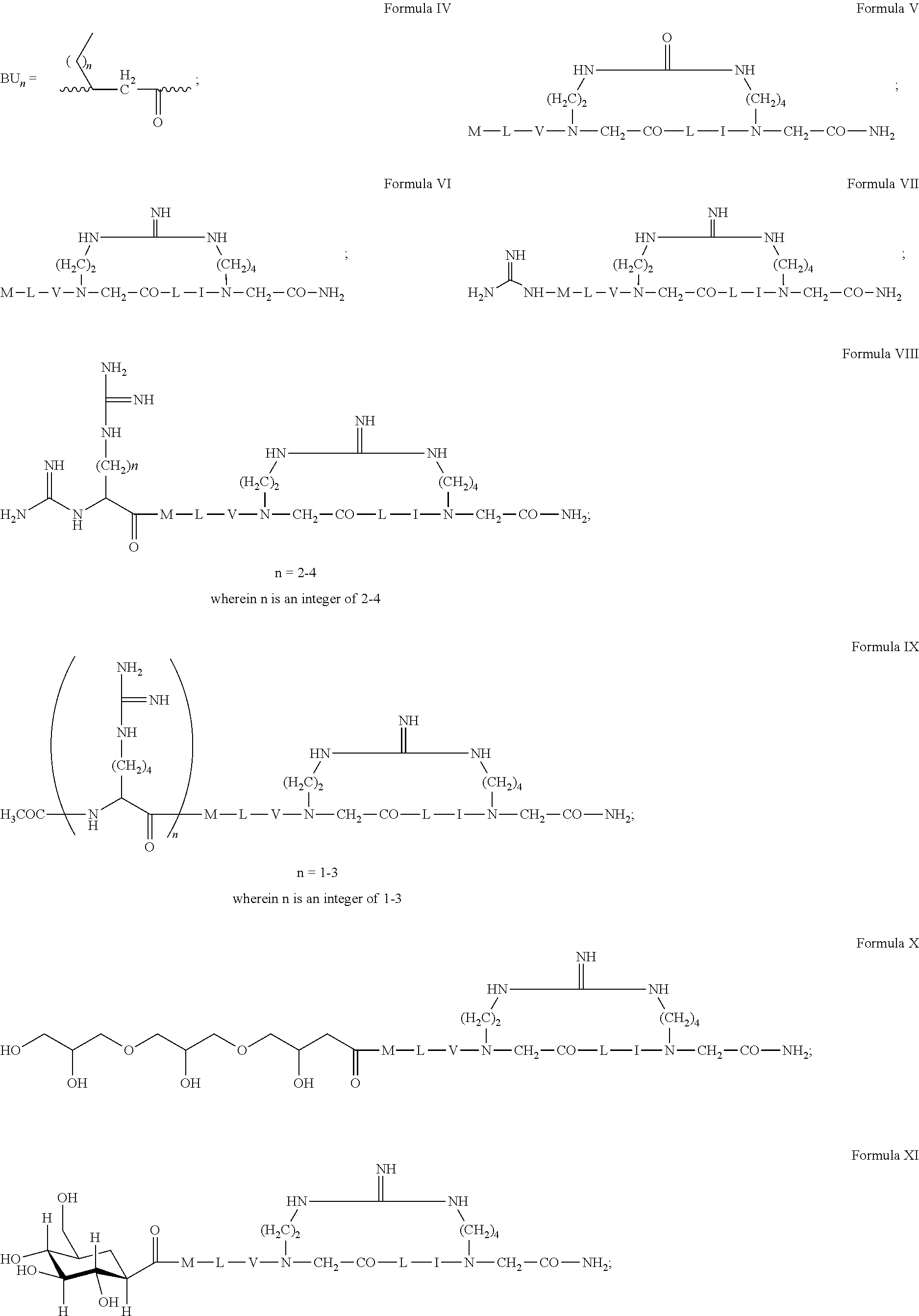

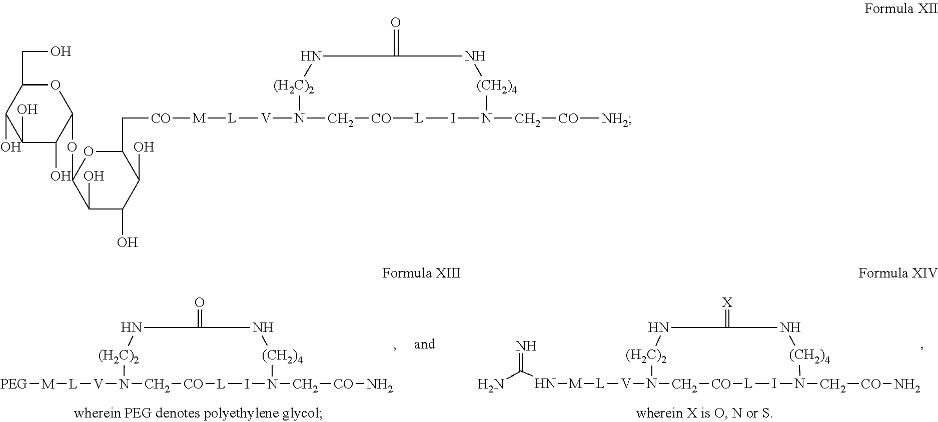

17. A pharmaceutical composition comprising at least one peptide according to claim 1, and optionally further comprising an excipient, carrier or diluent.

18. The pharmaceutical composition of claim 17 formulated for an administration mode selected from the group consisting of oral administration and parenteral administration.

19. A method of alleviation or treatment of a disease or disorder associated with expression of CCR2, comprising administering to a subject in need thereof, a pharmaceutical composition of claim 17.

20. The method of claim 19 wherein the disease or disorder associated with CCR2 expression is MS.
